(19) United States
(12) Patent Application Publication
(10) Pub. No.: US 2018/0100250 A1
(43) Pub. Date: Apr. 12, 2018

(54) Articles Comprising an Oil Capture Polymer

(71) Applicant: The Procter & Gamble Company, Cincinnati, OH (US)

(72) Inventors: Robin Lynn McKiernan, Mason, OH (US); Steven Daryl Smith, Fairfield, OH (US); Robert Joseph McChain, Cincinnati, OH (US); Jamie Lynn Dria, Deerfield Township, OH (US)

(73) Assignee: The Procter & Gamble Company

(21) Appl. No.: 15/729,695

(22) Filed: Oct. 11, 2017

Related U.S. Application Data
(60) Provisional application No. 62/406,424, filed on Oct. 11, 2016.

(51) Int. Cl. D01F 6/28; D01F 6/02; C08F 20/54; A61L 15/24

(52) U.S. Cl. CPC D01F 6/28; D01F 6/02; D10B 2509/026; A61L 15/24; C08F 20/54

(57) ABSTRACT

Articles, for example fibrous structures, that comprise one or more oil capture polymers and methods for making same.

### FIELD OF THE INVENTION

The present invention relates to articles, for example fibrous structures, that comprise one or more oil capture polymers and methods for making same.

### BACKGROUND OF THE INVENTION

Articles, for example fibrous structures, designed to adsorb hydrophilic soils, such as dirt, are known in the art. Further, articles that are designed to adsorb sebum using lauryl methacrylate/ethylene glycol methacrylate copolymer are also known. Further yet, an oil absorbing film made of methacrylic acid/C₉-C₂₄ alkyl methacrylate/C₁-C₂₄ methylacrylate is known in the art.

It has been found that a lot of household soils, such as cooking and kitchen soils, are hydrophobic. The known fibrous structures, articles, and films have proven to be ineffective at removing (adsorbing) these household hydrophobic soils from surfaces.

One problem with known articles, for example fibrous structures, is that they fail to effectively remove hydrophobic soils, such as cooking and kitchen soils, as shown by their L*a*b Sum A Values 85 or less as measured according to the Oil Capture Test Method described herein. For example, the polymers used in the known fibrous structures for removing soils are less hydrophobic, for example contain less hydrophobic moieties, and thus remove hydrophobic soils, such as oil, less effectively as measured by the Oil Capture Test Method described herein.

Accordingly, there is a need for an article, for example a fibrous structure that comprises an oil capture polymer that improves the removal/adsorption of household soils, such as hydrophobic household soils, for example hamburger grease, dirty motor oil, bacon grease, and other waxes compared to known articles, for example fibrous structures as measured according to the Oil Capture Test Method described herein.

### SUMMARY OF THE INVENTION

The present invention fulfills the need described above by providing an article, for example a fibrous structure, comprising an oil capture polymer and a method for making same.

One solution to the problem described above is an article, for example a fibrous structure, comprising an oil capture polymer such that the article, for example the fibrous structure, exhibits an L*a*b Sum A Value of greater than 85 and/or greater than 108.0 and/or greater than 110.0 as measured according to the Oil Capture Test Method described herein. To achieve this soil removal, the oil capture polymer of the article, for example the fibrous structure, is more hydrophobic, for example the polymer contains more hydrophobic moieties, compared to other soil adsorbing polymers of known articles in the art. Such oil capture polymers of the present invention are able to remove (adsorb) hydrophobic soils, such as cooking and kitchen soils, more readily and/or with less effort, for example without the need to apply a cleaning composition prior to contacting the soils with the article, for example the fibrous structure.

In one example of the present invention, an article, for example a fibrous structure, comprising an oil capture polymer, for example an oil capture polymer comprising two or more different monomeric units, such that the article, for example the fibrous structure, exhibits an L*a*b Sum A Value of greater than 85 as measured according to the Oil Capture Test Method, described herein, is provided.

In another example of the present invention, an article, for example a fibrous structure, comprising an oil capture polymer such that the article, for example the fibrous structure, exhibits an L*a*b Sum A Value of greater than 108.0 and/or greater than 110.0 as measured according to the Oil Capture Test Method, described herein, is provided.

In another example of the present invention, an article, for example a single- or multi-ply sanitary tissue product, such as a paper towel, comprising an oil capture polymer comprising two or more different monomeric units such that the article, for example the sanitary tissue product, exhibits an L*a*b Sum A Value of greater than 85 as measured according to the Oil Capture Test Method described herein, is provided.

In another example of the present invention, an article, for example a single- or multi-ply sanitary tissue product, such as a paper towel, comprising an oil capture polymer such that the article, for example the sanitary tissue product, exhibits an L*a*b Sum A Value of greater than 108.0 and/or greater than 110.0 as measured according to the Oil Capture Test Method described herein, is provided.

In yet another example of the present invention, an article, for example a hard surface cleaning pad, comprising a fibrous structure comprising an oil capture polymer comprising two or more different monomeric units such that the article, for example the hard surface cleaning pad, exhibits an L*a*b Sum A Value of greater than 85 as measured according to the Oil Capture Test Method described herein, is provided.

In yet another example of the present invention, an article, for example a hard surface cleaning pad, comprising a fibrous structure comprising an oil capture polymer such that the article, for example the hard surface cleaning pad, exhibits an L*a*b Sum A Value of greater than 108.0 and/or greater than 110.0 as measured according to the Oil Capture Test Method described herein, is provided.

In still another example of the present invention, a process for making an oil capture polymer comprising the steps of:
a. mixing two or more different oil capture polymer monomers in a solvent to form a monomer mixture;
b. adding a free radical initiator to the monomer mixture such that an oil capture polymer comprising two or more different monomeric units according to the present invention is produced, is provided.

In even still another example of the present invention, a process for making an article, for example a fibrous structure, comprising an oil capture polymer, the process comprising the steps of:
a. providing an article, for example a fibrous structure;
b. applying an oil capture polymer comprising two or more different monomeric units to a surface of the article, for example the fibrous structure, such that the article, for example the fibrous structure, exhibits an L*a*b Sum A Value of greater than 85 as measured according to the Oil Capture Test Method described herein, is provided.

In still yet another example of the present invention, a method for cleaning a surface comprising an oil, the method comprising the steps of:
a. providing an article, for example a fibrous structure, comprising an oil capture polymer comprising two or more different monomeric units; and
b. contacting the surface comprising an oil, for example a hydrophobic soil, such as a cooking and/or kitchen soil, with the article, for example the fibrous structure, such that the oil, for example the hydrophobic soil, is at least partially removed from the surface by the article, for example the fibrous structure, and wherein the article, for example the fibrous structure, exhibits an L*a*b Sum A Value of greater than 85 as measured according to the Oil Capture Test Method described herein, is provided.

In still another example of the present invention, a process for making an oil capture polymer comprising the steps of:
a. mixing two or more different oil capture polymer monomers in a solvent to form a monomer mixture;
b. adding a free radical initiator to the monomer mixture such that an oil capture polymer comprising two or more different monomeric units according to the present invention is produced, is provided.

In even still another example of the present invention, a process for making an article, for example a fibrous structure, comprising an oil capture polymer, the process comprising the steps of:
a. providing an article, for example a fibrous structure;
b. applying an oil capture polymer to a surface of the article, for example the fibrous structure, such that the article, for example the fibrous structure, exhibits an L*a*b Sum A Value of greater than 108.0 and/or greater than 110.0 as measured according to the Oil Capture Test Method described herein, is provided.

In still yet another example of the present invention, a method for cleaning a surface comprising an oil, the method comprising the steps of:
a. providing an article, for example a fibrous structure, comprising an oil capture polymer; and
b. contacting the surface comprising an oil, for example a hydrophobic soil, such as a cooking and/or kitchen soil, with the article, for example the fibrous structure, such that the oil, for example the hydrophobic soil, is at least partially removed from the surface by the article, for example the fibrous structure, and wherein the article, for example the fibrous structure, exhibits an L*a*b Sum A Value of greater than 108.0 and/or greater than 110.0 as measured according to the Oil Capture Test Method described herein, is provided.

In another example of the present invention, a system, for example for removing oil, such as hydrophobic soil, from a surface (the system may include instructions, such as in a kit comprising the article and the solution, for using the solution with the article in order to remove oil from a surface) comprising:
a. an article, for example a fibrous structure; and
b. a solution comprising an oil capture polymer, for example an oil capture polymer comprising two or more different monomeric units;
   such that when a surface comprising an oil, for example a hydrophobic soil, is contacted with the article and the solution, the oil is at least partially removed from the surface by the article and wherein the article, when the solution is present on the article, exhibits an L*a*b Sum A Value of greater than 85 and/or greater than 90 and/or greater than 108.0 and/or greater than 110.0 as measured according to the Oil Capture Test Method.

Accordingly, the present invention provides an article, for example a fibrous structure comprising an oil capture polymer such that the article, for example the fibrous structure, exhibits an L*a*b Sum A Value of greater than 85 and/or greater than 108.0 and/or greater than 110.0 as measured according to the Oil Capture Test Method described herein, products comprising such fibrous structures, processes for making such oil capture polymers, and methods for cleaning surfaces.

### BRIEF DESCRIPTION OF THE DRAWINGS

FIG. 1A is a schematic representation of a Swinging Arm Tester for testing fibrous structures;

FIG. 1B is a cross-section view of FIG. 1A taken along line 1B-1B;

FIG. 2 is a schematic representation of a fibrous structure tested according to the Oil Capture Test Method described herein showing locations on the fibrous structure to take measurements according to the Oil Capture Test Method.

### DETAILED DESCRIPTION OF THE INVENTION

### Definitions

“Article” as used herein means any solid matter, any liquid, such as an emulsion, containing solid matter and/or a film. Non-limiting examples of articles of the present invention include webs, wipes, wet wipes, sponges, foam structures, co-form materials, cotton pads, cotton combs, cotton swabs, dissolvable open cell foam, bar soap, laundry bars, laundry sheets, toothpastes, toothbrushes, floss, chewing gum, tooth strips, mops, liquid shampoos, liquid conditioners, mouthwashes, denture cleaning products. The liquid articles of the present invention include at least pieces and/or portions of solid matter, for example portions of webs. In one example, the article is a dry article. In one example, at least a portion of the article exhibits a basis weight of about 500 gsm or less, and/or about 300 gsm or less and/or about 150 gsm or less and/or about 100 gsm or less and/or to about 20 gsm and/or to about 30 gsm and/or to about 95 gsm. In yet another example, the article is a consumer goods article.

In one example, the article is selected from the group consisting of: towels, dryer sheets, filter media, wipes, sponges, mops, cleaning implements, door mats, car mats, disposable cloths, laundry sheets, paper towels, absorbent cores, scrubbing pads, brushes, facial tissue, dusters, and French press.

“Web” as used herein means a fibrous structure or a film.

“Fibrous structure” as used herein means a structure that comprises one or more fibrous filaments and/or fibers. In one example, a fibrous structure according to the present invention means an orderly arrangement of filaments and/or fibers within a structure in order to perform a function. In one example, a fibrous structure comprises inter-entangled filaments. Non-limiting examples of fibrous structures of the present invention include paper, fabrics (including woven, knitted, and non-woven), absorbent pads (for example for diapers or feminine hygiene products), cotton pads, and wipes.

Non-limiting examples of processes for making fibrous structures include known wet-laid processes, such as wet-laid papermaking processes, and air-laid processes, such as air-laid papermaking processes, meltblowing processes, spunbonding processes, solution spinning processes and other spinning processes. Wet-laid and/or air-laid papermaking processes and/or air-laid papermaking processes typically include a step of preparing a composition comprising a plurality of fibers that are suspended in a medium, either wet, more specifically aqueous medium, or dry, more specifically gaseous medium, such as air. The aqueous medium used for wet-laid processes is oftentimes referred to as a fiber slurry. The fiber composition is then used to deposit a plurality of fibers onto a forming wire or belt such that an embryonic fibrous structure is formed, after which drying and/or bonding the fibers together results in a fibrous structure. Further processing the fibrous structure may be carried out such that a finished fibrous structure is formed.

For example, in typical papermaking processes, the finished fibrous structure is the fibrous structure that is wound on the reel at the end of papermaking, and may subsequently be converted into a finished product, e.g. a sanitary tissue product.

“Fiber” and/or “Filament” as used herein means an elongate particulate having an apparent length greatly exceeding its apparent width, i.e. a length to diameter ratio of at least about 10. In one example, a “fiber” is an elongate particulate as described above that exhibits a length of less than 5.08 cm (2 in.) and a “filament” is an elongate particulate as described above that exhibits a length of greater than or equal to 5.08 cm (2 in.).

Fibers are typically considered discontinuous in nature. Non-limiting examples of fibers include wood pulp fibers and synthetic staple fibers such as polyester fibers.

Filaments are typically considered continuous or substantially continuous in nature. Filaments are relatively longer than fibers. Non-limiting examples of filaments include meltblown and/or spunbond filaments. Non-limiting examples of polymers, such as hydroxyl polymers, that can be spun into filaments include natural polymers, such as starch, starch derivatives, cellulose and cellulose derivatives, hemicellulose, hemicellulose derivatives, keratin, and synthetic polymers including, but not limited to polyvinyl alcohol filaments and/or polyvinyl alcohol derivative filaments, and thermoplastic polymer filaments, such as polyesters, nylons, polyolefins such as polypropylene filaments, polyethylene filaments, and biodegradable or compostable thermoplastic fibers such as polylactic acid filaments, polyhydroxyalkanoate filaments and polycaprolactone filaments. The filaments may be monocomponent or multicomponent, such as bicomponent filaments.

In one example, the article, for example a fibrous structure of the present invention comprises a hydroxyl polymer. For example, one or more filaments making up the fibrous structure may comprise a hydroxyl polymer, such as a hydroxyl polymer selected from the group consisting of: polyvinyl alcohol, cellulose, carboxymethylcellulose, chitin, chitosan, starch, starch derivatives, keratin, and mixtures thereof.

In one example, the article, for example a fibrous structure of the present invention comprises an amine moiety, such as a primary, secondary, and/or tertiary amine.

In one example of the present invention, “fiber” refers to papermaking fibers. Papermaking fibers useful in the present invention include cellulosic fibers commonly known as wood pulp fibers. Applicable wood pulps include chemical pulps, such as Kraft, sulfite, and sulfate pulps, as well as mechanical pulps including, for example, groundwood, thermomechanical pulp and chemically modified thermomechanical pulp. Chemical pulps, however, may be preferred since they impart a superior tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified web. Also applicable to the present invention are fibers derived from recycled paper, which may contain any or all of the above categories as well as other non-fibrous materials such as fillers and adhesives used to facilitate the original papermaking.

In addition to the various wood pulp fibers, other cellulosic fibers such as cotton linters, rayon, lyocell, trichomes, and bagasse can be used in this invention. Other sources of cellulose in the form of fibers or capable of being spun into fibers include grasses and grain sources.

In one example, the fibrous structure of the present invention may comprise filaments, such as polypropylene filaments, and fibers, such as pulp fibers, such as a co-formed fibrous structure. The pulp fibers may be the article-forming components that comprise a durably bonded oil capture polymer.

“Dry article” as used herein means an article that comprises less than 30% and/or less than 20% and/or less than 15% and/or less than 10% and/or less than 7% and/or less than 5% and/or less than 3% and/or less than 2% and/or less than 1% and/or less than 0.5% by weight of water (moisture) as measured according to the Water Content Test Method described herein.

“Dry web” as used herein means a web that comprises less than 30% and/or less than 20% and/or less than 15% and/or less than 10% and/or less than 7% and/or less than 5% and/or less than 3% and/or less than 2% and/or less than 1% and/or less than 0.5% by weight of water (moisture) as measured according to the Water Content Test Method described herein.

“Dry fibrous structure” as used herein means a fibrous structure that comprises less than 30% and/or less than 20% and/or less than 15% and/or less than 10% and/or less than 7% and/or less than 5% and/or less than 3% and/or less than 2% and/or less than 1% and/or less than 0.5% by weight of water (moisture) as measured according to the Water Content Test Method described herein.

“Sanitary tissue product” as used herein means a soft, low density (i.e. <about 0.15 g/cm³) web useful as a wiping implement for post-urinary and post-bowel movement cleaning (toilet tissue), for otorhinolaryngological discharges (facial tissue), multi-functional absorbent and cleaning uses (absorbent towels), and folded sanitary tissue products such as napkins and/or facial tissues including folded sanitary tissue products dispensed from a container, such as a box. The sanitary tissue product may be convolutedly wound upon itself about a core or without a core to form a sanitary tissue product roll.

In one example, the sanitary tissue product of the present invention comprises a fibrous structure according to the present invention.

The sanitary tissue products of the present invention may exhibit a basis weight between about 10 g/m² to about 120 g/m² and/or from about 15 g/m² to about 110 g/m² and/or from about 20 g/m² to about 100 g/m² and/or from about 30 to 90 g/m². In addition, the sanitary tissue product of the present invention may exhibit a basis weight between about 40 g/m² to about 120 g/m² and/or from about 50 g/m² to about 110 g/m² and/or from about 55 g/m² to about 105 g/m² and/or from about 60 to 100 g/m².

The sanitary tissue products of the present invention may exhibit a density (measured at 95 g/in²) of less than about 0.60 g/cm³ and/or less than about 0.30 g/cm³ and/or less than about 0.20 g/cm³ and/or less than about 0.10 g/cm³ and/or less than about 0.07 g/cm³ and/or less than about 0.05 g/cm³ and/or from about 0.01 g/cm³ to about 0.20 g/cm³ and/or from about 0.02 g/cm³ to about 0.10 g/cm³.

The sanitary tissue products of the present invention may be in the form of sanitary tissue product rolls. Such sanitary tissue product rolls may comprise a plurality of connected, but perforated sheets of fibrous structure, that are separably dispensable from adjacent sheets. In one example, one or more ends of the roll of sanitary tissue product may comprise an adhesive and/or dry strength agent to mitigate the loss of fibers, especially wood pulp fibers from the ends of the roll of sanitary tissue product.

The sanitary tissue products of the present invention may comprises additives such as softening agents, temporary wet strength agents, permanent wet strength agents, bulk softening agents, lotions, silicones, wetting agents, latexes, especially surface-pattern-applied latexes, dry strength agents such as carboxymethylcellulose and starch, and other types of additives suitable for inclusion in and/or on sanitary tissue products.

“Film” refers to a sheet-like material wherein the length and width of the material far exceed the thickness of the material.

“Hard surface” refers to any surface of a non-supple material. Non-limiting examples of hard surfaces are typically found in and around houses like bathrooms, kitchens, basements and garages, e.g., floors, walls, tiles, windows, countertops, sinks, showers, shower curtains, shower doors, wash basins, dishes, bath fixtures, kitchen fixtures, appliances, toilets, bath tubs, mirrors, glass surfaces, wood surfaces, tiles, linoleum, automotive surfaces (interior and exterior), windshields, furniture, laminates, granite, synthetic solid surfaces, such as Corian® by DuPont, and fittings and the like made of different materials like ceramic, enamel, painted and un-painted concrete, plaster, bricks, vinyl, no-wax vinyl, linoleum, melamine, Formica®, glass, any plastics, metals, chromed surface and the like. The term “hard surface” as used herein also includes household appliances including, but not limited to, washing machines, automatic dryers, refrigerators, freezers, ovens, microwave ovens, dishwashers and the like.

“Hydrophilic” as used herein means a surface is wettable by aqueous fluids deposited thereon. Hydrophilicity and wettability are typically defined in terms of contact angle and the surface tension of the fluids and surfaces involved. This is discussed in detail in the American Chemical Society publication entitled Contact Angle, Wettability and Adhesion, edited by Robert F. Gould (Copyright 1964) which is hereby incorporated by reference. A surface is said to be wetted by an aqueous fluid (hydrophilic) when the fluid tends to spread spontaneously across the surface. Conversely, a surface is considered to be “hydrophobic” if the aqueous fluid does not tend to spread spontaneously across the surface.

In one example, “hydrophilic” and “hydrophobic” have meanings well established in the art with respect to the contact angle of a drop of water on the surface of a material. Thus, a material having a contact angle of greater than 75° is considered hydrophobic, and a material having a contact angle of 75° or less is considered hydrophilic. Absolute values of hydrophobocity/hydrophilicity are not generally important, but relative values are.

“Basis Weight” as used herein is the weight per unit area of a sample reported in lbs/3000 ft² or g/m² and is measured according to the Basis Weight Test Method described herein.

“By weight of water” or “water content” or “by weight of moisture” or “moisture content” means the amount of water (moisture) present in an article measured according to the Water Content Test Method described herein immediately after the article has been conditioned in a conditioned room at a temperature of 73° F.±4° F. (about 23° C.±2.2° C.) and a relative humidity of 50%±10% for 2 hours.

“Machine Direction” or “MD” as used herein means the direction parallel to the flow of The fibrous structure through The fibrous structure making machine and/or sanitary tissue product manufacturing equipment.

“Cross Machine Direction” or “CD” as used herein means the direction parallel to the width of The fibrous structure making machine and/or sanitary tissue product manufacturing equipment and perpendicular to the machine direction.

“Ply” as used herein means an individual, integral fibrous structure.

“Plies” as used herein means two or more individual, integral fibrous structures disposed in a substantially contiguous, face-to-face relationship with one another, forming a multi-ply fibrous structure and/or multi-ply sanitary tissue product. It is also contemplated that an individual, integral fibrous structure can effectively form a multi-ply fibrous structure, for example, by being folded on itself.

“Monomeric unit” as used herein is a constituent unit (sometimes referred to as a structural unit) of a polymer.

### Article

The article, for example a fibrous structure, of the present invention comprises an oil capture polymer. The article may be in the form of a wet article or a dry article, for example a wet fibrous structure, such as a premoistened fibrous structure and/or a fibrous structure comprising a liquid composition, or a dry fibrous structure, or a combination wet and dry article. The article may be designed to be used wet and/or dry.

In one example, the article comprises a web. In another example, the article comprises a nonwoven material such as a paper towel, napkins, a dryer sheet, a laundry sheet, a filter medium, a wipe, a toilet tissue, a facial tissue, surgical gowns, and face masks. In still another example, the article comprises a woven material such as a towel, wash cloths, garments, sports apparel, and gloves. In even other examples, the article of the present invention is disposable. In still another example, the article of the present invention comprises sponges, mops, cleaning implements such as cleaning pads, for example Swiffer® cleaning pads, door mats, car mats, disposable cloths, absorbent cores for use in various absorbent products such as diapers and feminine hygiene products, scrubbing pads, brushes, and dusters such as Swiffer® dusters.

When the article comprises a web, the web may comprise a fibrous structure. The fibrous structure may be a dry fibrous structure.

The fibrous structure of the present invention may comprise a plurality of pulp fibers, such as wood pulp fibers. Further, the fibrous structure of the present invention may comprise a single-ply or multi-ply sanitary tissue product, such as a paper towel.

In still another example, the fibrous structure of the present invention may comprise a plurality of filaments. The filaments may be inter-entangled to form the fibrous structure.

In even still another example, the fibrous structure of the present invention may comprise a plurality of filaments and a plurality of fibers, for example wood pulp fibers.

In another example, the article of the present invention may comprise a web, for example a fibrous structure, in the form of a cleaning pad suitable for use with a cleaning device, such as a floor cleaning device, for example a Swiffer® cleaning pad or equivalent cleaning pads.

In still another example, the article of the present invention may comprise a foam structure.

The article of the present invention may be used to clean various surfaces, such as hard surfaces. Non-limiting examples of hard surfaces include kitchen countertops, appliances, dishes, pots, pans, sinks, floors, tables, outdoor furniture, cars, trucks, windows, mirrors, blinds, fans, lamps, lights, televisions, tile, glass, linoleum, tires, wheels, rims, metal surfaces, concrete surfaces, laminates, paintings, photographs, banisters, doors, eyeglasses, bathroom surfaces including toilet, toilet bowls, showers, teeth, and tubs, and the like.

The article of the present invention may be used alone or in combination with other components, such as a liquid, to clean the surfaces to be cleaned. The liquid may help activate and/or soften the oil capture polymer such that it is capable of removing (adsorbing) the hydrophobic soils, for example oils, such as cooking and/or kitchen soils.

The article of the present invention comprises an oil capture polymer. The oil capture polymer may be present in and/or on the article at a level of greater than 0% and/or greater than 0.05% and/or greater than 0.1% and/or greater than 0.2% and/or greater than 0.5% and/or greater than 1% and/or greater than 1.5% and/or greater than 2.0% and/or to about 10% and/or less than 10% and/or less than 7% and/or less than 5% and/or less than 3% and/or less than 2% and/or less than 1% by weight of the article. In one example, the oil capture polymer is present in and/or on the article at a level of from about 0.1% to about 7% and/or from about 0.2% to about 5% by weight of the article.

In another example of the present invention, an article may comprise an oil capture polymer at a level of greater than 2 pounds/ton (#/ton) and/or greater than 3 #/ton and/or greater than 4 #/ton and/or greater than 8 #/ton and/or less than 200 #/ton and/or less than 100 #/ton and/or less than 50 #/ton and/or less than 20 #/ton and/or less than 15 #/ton and/or less than 10 #/ton by weight of the article. The level of oil capture polymer present in and/or on an article as used herein according to the present invention is in terms of active solids basis of the oil capture polymer. In one example, the oil capture polymer is present in and/or on the article at a level of from about 2 #/ton to about 200 #/ton and/or from about 4 #/ton to about 100 #/ton by weight of the article.

The article may comprise other ingredients in addition to the oil capture polymer, for example a surfactant. The surfactant may be present in the article at a level of from about 0.01% to about 0.5% by weight of the article. Non-limiting examples of a suitable surfactant include C₈₋₁₆ alkyl polyglucoside, cocoamido propyl sulfobetaine or mixtures thereof.

In one example, the article comprises a signal, such as a dye and/or pigment that becomes visible or becomes invisible to a consumer's eye when the article adsorbs soil and/or when a oil capture polymer present in and/or on the article adsorbs soil. In another example, the signal may be a difference in texture of the article or a difference in the physical state of the article, for example the article dissolves and/or vaporizes when the article adsorbs soil.

In another example, the oil capture polymer may be present in and/or on an article in a pattern, such as a non-random repeating pattern composing lines and or letters/words, and/or present in and/or on regions of different density, different basis weight, different elevation and/or different texture of the article.

In one example, the article of the present invention exhibits an L*a*b Sum A Value of greater than 85 and/or greater than 90 and/or greater than 95 and/or greater than 100 and/or greater than 108.0 and/or greater than 110.0 and/or greater than 120 and/or greater than 130 and/or greater than 140 and/or greater than 150 as measured according to the Oil Capture Test Method described herein.

### Oil Capture Polymers

The oil capture polymers of the present invention comprise one or more oil capture polymer monomeric units that are derived from corresponding monomers capable of forming an oil capture polymer.

In one example of the present invention, an oil capture polymer of the present invention is a polymer that when in contact with, such as applied to a surface of, during, prior to, or after use, an article, such as a fibrous structure, results in the fibrous structure exhibiting a greater L*a*b Sum A Value than the fibrous structure void of the polymer, in other words, when the polymer doesn't come into contact with the article, during, prior to, or after use. In one example of the present invention, an oil capture polymer of the present invention is a polymer that when in contact with, such as applied to a surface of, during, prior to, or after use, an article, such as a fibrous structure, results in the fibrous structure exhibiting a greater L*a*b Sum A Value of greater than 85 and/or greater than 90 and/or greater than 95 and/or greater than 100 and/or greater than 108.0 and/or greater than 110.0 and/or greater than 120 and/or greater than 130 and/or greater than 140 and/or greater than 150 as measured according to the Oil Capture Test Method described herein.

Monomers Capable of Forming an Oil Capture Polymer

The oil capture polymers of the present invention comprise one or more, in one example two or more, different types of monomeric units capable of forming an oil capture polymer derived from corresponding oil capture polymer monomers. As a result, the oil capture polymers of the present invention can be referred to as homopolymers or copolymers including terpolymers and higher. In another example, the oil capture polymer of the present invention is a random copolymer. In another example, the oil capture polymer of the present invention is a homopolymer.

In one example, an oil capture polymer of the present invention comprises a polymer comprising a monomeric unit selected from the group consisting of: acrylamide monomeric units or derivatives thereof, methacrylamide monomeric units or derivatives thereof, and mixtures thereof.

In one example, an oil capture polymer of the present invention comprises greater than 50% and/or greater than 60% and/or greater than 70% and/or greater than 80% and/or at least 85% and/or at least 90% and/or at least 95% and/or at least 98% and/or at least 99% and/or 100% by weight of the monomeric unit derived from an acrylamide (AAM), acrylamide derivative, methacrylamide (MAAM), methacrylamide derivative, and mixtures thereof.

In one example, the acrylamide derivatives of the present invention are selected from the group consisting of: symmetrical or asymmetrical, di-N-alkyl substituted C₁-C₁₀₀ acrylamide derivatives, and mixtures thereof. In another example, the acrylamide derivatives of the present invention are selected from the group consisting of: N,N-dimethylacrylamide (NDMAAM), octadecylacrylamide (ODAA), N-2-ethylhexyl acrylamide, N-dodecylacrylamide, N,N-dioctadecylacrylamide, N,N-di-2-ethylhexyl acrylamide, N,N-didodecylacrylamide, N-methyl-N-ethyl-acrylamide, N-methyl-N-octadecyl-acrylamide, N-methyl-N-2-ethylhexyl-acrylamide, acrylomorpholine, and mixtures thereof.

In one example, the methacrylamide derivatives of the present invention are selected from the group consisting of: symmetrical or asymmetrical, di-N-alkyl substituted C₁-C₁₀₀ methacrylamide derivatives, and mixtures thereof. In another example, the methacrylamide derivatives of the present invention are selected from the group consisting of: N-[3-(dimethylamino)propyl]methacrylamide (DMAPMA), N-octadecylmethacrylamide, N-2-ethylhexyl methacrylamide, N-dodecyl methacrylamide, N,N-dioctadecylmethacrylamide, N,N-di-2-ethylhexyl methacrylamide, N,N-didodecyl methacrylamide, N-methyl-N-ethyl-methacrylamide, N-methyl-N-octadecyl-methacrylamide, N-methyl-N-2-ethylhexyl-methacrylamide, and mixtures thereof.

In one example, an oil capture polymer of the present invention comprises a monomeric unit derived from N,N-dimethylacrylamide and one or more other monomeric units derived from acrylamide or methacrylamide derivatives selected from the group consisting of: N-[3-(dimethylamino)propyl]methacrylamide, N-octadecylacrylamide, N-2-ethylhexyl acrylamide, N-dodecylacrylamide, N-octadecylmethacrylamide, N-2-ethylhexyl methacrylamide, N-dodecyl methacrylamide, N,N-dioctadecylacrylamide, N,N-di-2-ethylhexyl acrylamide, N,N-didodecylacrylamide, N,N-dioctadecylmethacrylamide, N,N-di-2-ethylhexyl methacrylamide, N,N-didodecyl methacrylamide, N-methyl-N-ethyl-acrylamide, N-methyl-N-ethyl-methacrylamide, N-methyl-N-octadecyl-acrylamide, N-methyl-N-octadecyl-methacrylamide, N-methyl-N-2-ethylhexyl-acrylamide, N-methyl-N-2-ethylhexyl-methacrylamide, and mixtures thereof.

In one example, the oil capture polymer comprises a cationic monomeric unit. Non-limiting examples of cationic monomeric units suitable for the present invention include cationic monomeric units derived from cationic monomers selected from the group consisting of: N,N-(dialkylamino-ω-alkyl)amides of α,β-monoethylenically unsaturated carboxylic acids, such as N,N-dimethylaminomethylacrylamide or -methacrylamide, 2-(N,N-dimethylamino)ethylacrylamide or -methacrylamide, 3-(N,N-dimethylamino)propylacrylamide or -methacrylamide, and 4-(N,N-dimethylamino)butylacrylamide or -methacrylamide, α,β-monoethylenically unsaturated amino esters such as 2-(dimethylamino)ethyl acrylate (DMAA), 2-(dimethylamino)ethyl methacrylate (DMAM), 3-(dimethylamino)propyl methacrylate, 2-(tert-butylamino)ethyl methacrylate, 2-(dipentylamino)ethyl methacrylate, and 2(diethylamino)ethyl methacrylate, vinylpyridines, vinylamine, vinylimidazolines, monomers that are precursors of amine functions such as N-vinylformamide, N-vinylacetamide, which give rise to primary amine functions by simple acid or base hydrolysis, acryloyl- or acryloyloxyammonium monomers such as trimethylammonium propyl methacrylate chloride, trimethylammonium ethylacrylamide or -methacrylamide chloride or bromide, trimethylammonium butylacrylamide or -methacrylamide methyl sulfate, trimethylammonium propylmethacrylamide methyl sulfate, (3-methacrylamidopropyl)trimethylammonium chloride (MAPTAC), (3-methacrylamidopropyl)trimethylammonium methyl sulphate (MAPTA-MES), (3-acrylamidopropyl)trimethylammonium chloride (APTAC), methacryloyloxyethyl-trimethylammonium chloride or methyl sulfate, and acryloyloxyethyltrimethylammonium chloride; 1-ethyl-2-vinylpyridinium or 1-ethyl-4-vinylpyridinium bromide, chloride or methyl sulfate; N,N-dialkyldiallylamine monomers such as N,N-dimethyldiallylammonium chloride (DADMAC); polyquaternary monomers such as dimethylaminopropylmethacrylamide chloride and N-(3-chloro-2-hydroxypropyl)trimethylammonium (DIQUAT or DQ) and 2-hydroxy-N¹-(3-(2((3-methacrylamidopropyl)dimethylammino)-acetamido)propyl)-N¹, N¹, N³, N³, N³-pentamethylpropane-1,3-diaminium chloride (TRIQUAT or TQ), and mixtures thereof. In one example, the cationic monomeric unit comprises a quaternary ammonium monomeric unit, for example a monoquaternary ammonium monomeric unit, a diquaternary ammonium monomeric unit and a triquaternary monomeric unit. In one example, the cationic monomeric unit is derived from MAPTAC. In another example, the cationic monomeric unit is derived from DADMAC. In still another example, the cationic monomeric unit is derived from TQ.

In one example, the cationic monomeric units are derived from cationic monomers selected from the group consisting of: dimethylaminoethyl (meth)acrylate, dimethylaminopropyl (meth)acrylate, di-tert-butylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine and vinyl imidazole, and mixtures thereof.

In one example, these cationic monomer units are included in the oil capture polymer to permit increased monomeric unit levels of hydrophobic monomeric units, such as ODAA, while maintaining water solubility of the oil capture polymer.

In one example, the oil capture polymer of the present invention comprises a monomeric unit derived from N,N-dimethylacrylamide. In one example, the oil capture polymer comprises greater than 75% and/or greater than 80% and/or greater than 85% and/or at least 90% and/or at least 95% and/or at least 98% and/or at least 99% and/or 100% by weight of a monomeric unit derived from N,N-dimethylacrylamide.

In one example, the oil capture polymer of the present invention is a homopolymer comprising a monomeric unit derived from a compound selected from the group consisting of: acrylamide, acrylamide derivatives, methacrylamide, and methacrylamide derivatives. In one example, the oil capture polymer of the present invention is a homopolymer of N,N-dimethylacrylamide.

In one example, the oil capture polymer comprises greater than 50% and/or greater than 75% and/or greater than 80% and/or greater than 85% and/or at least 90% and/or at least 95% and/or at least 98% and/or at least 99% by weight of a monomeric unit derived from N,N-dimethylacrylamide and less than 20% and/or less than 15% and/or 10% or less and/or 5% or less and/or 4% or less and/or 2% or less and/or 1% or less and/or 0.5% or less but greater than 0% by weight of a monomeric unit derived from N-octadecylacrylamide.

In another example, the oil capture polymer comprises greater than 75% and/or greater than 80% and/or greater than 85% and/or at least 90% and/or at least 95% and/or at least 98% and/or at least 99% by weight of a monomeric unit derived from N,N-dimethylacrylamide and less than 20% and/or less than 15% and/or 10% or less and/or 5% or less and/or 4% or less and/or 2% or less and/or 1% or less and/or 0.5% or less but greater than 0% by weight of a monomeric unit derived from N-[3-(dimethylamino)propyl]methacrylamide.

In one example, the oil capture polymer comprises greater than 75% and/or greater than 80% and/or greater than 85% and/or at least 90% and/or at least 95% and/or at least 98% and/or at least 99% by weight of a monomeric unit derived from N,N-dimethylacrylamide and less than 20% and/or less than 15% and/or 10% or less and/or 5% or less and/or 4% or less and/or 2% or less and/or 1% or less and/or 0.5% or less but greater than 0% by weight of a monomeric unit derived from N-2-ethylhexyl acrylamide.

In one example, the oil capture polymer comprises greater than 75% and/or greater than 80% and/or greater than 85% and/or at least 90% and/or at least 95% and/or at least 98% and/or at least 99% by weight of a monomeric unit derived from N,N-dimethylacrylamide and less than 20% and/or less than 15% and/or 10% or less and/or 5% or less and/or 4% or less and/or 2% or less and/or 1% or less and/or 0.5% or less but greater than 0% by weight of a monomeric unit derived from N-dodecylacrylamide.

In one example, post-polymerization modifications can be made to the oil capture polymer. Non-limiting examples of such post-polymerization modifications include, but are not limited to, conversion of tertiary amines to amine oxides or quaternary amines.

In one example, the oil capture polymer of the present invention is water soluble.

In one example, the oil capture polymer of the present invention may be mixed solvent dispersible to permit application to articles, such as fibrous structures.

### Non-Limiting Synthesis Example for Making an Oil Capture Polymer

An example of an oil capture polymer according to the present invention is made as follows. Into a reaction vessel, place monomers (NDMAAM available from Aldrich, ODAA available from Polysciences, DMAPMA available from Aldrich, N-2-ethylhexyl acrylamide available from Aurora Fine Chemicals, LLC, (San Diego, Calif., 92126) and N-dodecyl acrylamide available from TCI) and solvent (ethyl acetate available from EMD Chemicals was used as the solvent for all the samples) in the amounts listed in Table 1 below. The reaction vessel is closed and heated to the temperature (Rxn Temp ° C.) also listed in Table 1 below. Once at temperature, the reaction vessel is opened and the contents are sparged with an inert gas, such as but not limited to nitrogen or argon, for approximately four minutes utilizing a fritted gas dispersion tube. During the sparge, a free radical initiator V-67 (2,2′-azobis(2-methylbutyronitrile) available from DuPont) or V-70 (2,2′-azobis(2,4-dimethyl-4-methoxyvaleronitrile) available from Wako) is added to the reaction vessel. Except for Sample 2, the free radical initiator used is V-67. Sample 2's free radical is V-70. The amount of free radical initiator is set forth in Table 1 below. The free radical initiator is added at approximately ½ sparge time to ensure the free radical initiator also undergoes some sparge. The contents are then sealed and kept at the temperature previously listed in Table 1 below for a time also listed in Table 1 below. After the elapsed time (Rxn Time) has transpired, the resulting polymer solution is cooled to 23° C.±2.2° C. then precipitated in a co-solution of ethyl acetate and hexanes from 40% to 80% ethyl acetate, such as 50% ethyl acetate and 50% hexane by volume. The precipitate is isolated from the solvent mixture and dried. Once dried the product can be used as is or can be dissolved in solvent system of choice per application. The solvent may be water. Polymer Sample 1 in Table 1 below is a homopolymer NDMAAMe. Polymer Samples 2-12 in Table 1 below are examples of soil capture polymers comprising two or more different monomeric units.

TABLE 1

| | | Amount | | Amount | Solvent | Initiator | Rxn | Rxn |
|---|---|---|---|---|---|---|---|---|
| Polymer | | Monomer 1 | | Monomer 2 | Amount | Amount | Temp | Time |
| Sample | Monomer 1 | (g) | Monomer 2 | (g) | (g) | (g) | ° C. | (hours) |
| 1 | NDMAAM | 20.276 | — | — | 379.4997 | 0.40641 | 50 | 53 |
| 2 | NDMAAM | 99.67 | ODAA | 0.3308 | 299.56 | 0.463 | 30 | 70 |
| 3 | NDMAAM | 140.194 | ODAA | 1.358 | 2544.9 | 2.83 | 50 | 70 |
| 4 | NDMAAM | 19.617 | ODAA | 0.6398 | 383.8437 | 0.40093 | 50 | 53 |
| 5 | NDMAAM | 47.507 | DMAPMA | 2.522 | 252 | 1.013 | 60 | 24 |
| 6 | NDMAAM | 45.007 | DMAPMA | 5.046 | 254 | 1.012 | 60 | 24 |
| 7 | NDMAAM | 49.011 | C8* | 1.024 | 250 | 1 | 60 | 24 |
| 8 | NDMAAM | 47.51 | C8* | 2.522 | 250 | 1 | 60 | 24 |
| 9 | NDMAAM | 45.034 | C8* | 5.007 | 250 | 1 | 60 | 24 |
| 10 | NDMAAM | 49.041 | C12** | 1.012 | 250 | 1 | 60 | 24 |
| 11 | NDMAAM | 45.526 | C12** | 2.523 | 250 | 1 | 60 | 24 |
| 12 | NDMAAM | 45.016 | C12** | 5.018 | 250 | 1 | 60 | 24 |

*C8 is N-2-ethylhexyl acrylamide
**C12 is N-dodecyl acrylamide

L*a*b Sum A Values of inventive fibrous structure samples comprising the polymer samples from Table 1 above are shown in Table 2 below. Fibrous Structure Sample 1 comprises a homopolymer NDMAAM. Fibrous Structure Samples 2-12 comprise an oil capture polymer comprising two or more different monomeric units. Fibrous Structure Sample Water is a fibrous structure void of an oil capture polymer of the present invention and only contains 6.7 mL of distilled water. The fibrous structure samples are prepared and test according to the Oil Capture Test Method described herein.

TABLE 2

| Fibrous | | | | | |
|---|---|---|---|---|---|
| Structure | | Monomer 1 | Monomer 2 | Monomer 2 | L*a*b |
| Sample | Monomer 1 | Wt % | (hydrophobic) | Wt % | Sum A |
| 1 | NDMAAM | 100% | — | — | 107.9 |
| 2 | NDMAAM | 99.67% | ODAA | 0.33% | 129.1 |
| 3 | NDMAAM | 99% | ODAA | 1% | 141.4 |
| 4 | NDMAAM | 96.8% | ODAA | 3.2% | 159.1 |
| 5 | NDMAAM | 95% | DMAPMA | 5% | 116.3 |
| 6 | NDMAAM | 90% | DMAPMA | 10% | 151.5 |
| 7 | NDMAAM | 98% | C8* | 2% | 90.7 |
| 8 | NDMAAM | 95% | C8* | 5% | 111.8 |
| 9 | NDMAAM | 90% | C8* | 10% | 110.5 |
| 10 | NDMAAM | 98% | C12** | 2% | 98 |
| 11 | NDMAAM | 95% | C12** | 5% | 115.9 |
| 12 | NDMAAM | 90% | C12** | 10% | 100.3 |
| Water | — | — | — | — | 81.2 |
| (Control) | | | | | |

*C8 is N-2-ethylhexyl acrylamide
**C12 is N-dodecyl acrylamide

### Test Methods

Unless otherwise specified, all tests described herein including those described under the Definitions section and the following test methods are conducted on samples that have been conditioned in a conditioned room at a temperature of 23° C.±2° C. and a relative humidity of 50%±2% for a minimum of 2 hours prior to the test. The samples tested are “usable units.” “Usable units” as used herein means sheets, flats from roll stock, pre-converted flats, sheet, and/or single or multi-compartment products. Do not test samples that have defects such as wrinkles, tears, holes, and like. Samples conditioned as described herein are considered dry samples (such as “dry filaments”) for testing purposes. All instruments are calibrated according to manufacturer's specifications.

### Oil Capture Test Method

The effectiveness of an article, for example a fibrous structure, to remove an oil, for example a hydrophobic soil, from a surface, such as a hard surface, is measured according to the following Oil Capture Test Method as follows.

1. Test Sample Preparation

First, an article, for example a fibrous structure is prepared for the test. Preparation of the article, for example fibrous structure, is as follows.

Cut 3 test samples from an article, for example a fibrous structure to be tested, to 6 inch by 3.5 inch rectangles, as needed. Label the test samples with a ball-point or equivalent marker. Measure the weight of each test sample to within ±10 mg. Determine the average weight of the 3 test samples and record average weight (Weight_{Average}). If a test sample has been pre-treated, such as with a polymer, for example an oil capture polymer, it can be tested without further addition of any polymer solution. If the test sample has not been pre-treated, such as with a polymer, for example an oil capture polymer, place the test sample on an elevated lattice (23.75 inch by 47.75 inch polystyrene light panel manufactured by Plaskolite, Inc., Columbus, Ohio, available from Home Depot as model number 1425005a or equivalent lattice) and slowly pipette 6.7 mL of 0.50% polymer solution (as prepared below in section 3) onto each non pre-treated test sample and after application of all the polymer solution, the test samples are allowed to dry for at least 4 hours typically overnight on the lattice. Alternately, if the test sample is untreated and the starting polymer solution is less than 0.50%, apply additional aliquots to the test sample so that the final polymer add on level is equivalent to 6.7 mL of a 0.50% polymer solution. This may require multiple aliquots with at least 1.5 hours between additional aliquots to prevent oversaturation and a final drying of at least 4 hours typically overnight on the lattice. For comparison, the control is made with 6.7 mL of distilled water and then allowed to dry in the same manner as the above samples.

2. Determination of Percent Solids

If a test sample is untreated and the polymer solution concentration is unknown, determine percent solids of the polymer solution prior to applying the polymer solution to the untreated test sample as follows: Record weight of empty weigh pan (VWR disposable aluminum crinkle dish with tab, VWR Catalog #24333-010) to within ±0.1 mg, WeightPan. Place aliquot (2.5±0.5 grams) of polymer solution into the pan and record the weight to within ±0.1 mg, WeightPan+Polymer Solution. Place pan with polymer solution into ventilated oven set at 80° C., uncovered for at least 12 hours. Remove pans from oven, cool to room temperature (73° F.±3.5° F. and a relative humidity of less than 70%), and record the weight of the pan to within ±0.1 mg, WeightPan+Polymer Solids. Calculate percent solids as follows:

$$PercentSolids ( \% ) = ( \frac{{Weight}_{Pan + PolymerSolid} - {Weight}_{Pan}}{{Weight}_{Pan + PolymerSolution} - {Weight}_{Pan}} ) * 100 \%$$

3. Preparation of 0.50% Polymer Solution

If a test sample is untreated and the polymer concentration is greater than 0.50% by mass then prepare a 0.50% polymer solution as follows: Tare a receiving vessel of sufficient capacity to contain the resultant 0.50% polymer solution. Add the desired amount of the polymer or polymer solution to the tared vessel and record the weight, WeightPolymer Solution. Dilute the polymer or polymer solution to 0.50% with deionized water and record the weight, WeightPolymer Solution+Water. Cap the diluted solution and allow to sit for 24 hours with occasional agitation prior to use to ensure polymer dissolution. Calculate concentration as follows:

$$Concentration ( \% ) = \frac{{Weight}_{PolymerSolution} * PercentSolids}{{Weight}_{PolymerSolution + Water}}$$

4. Method to Polymerize Oil

To prepare the polymerized oil, a hydrophobic soil, to be applied to a clean ceramic tile as described below, one liter of Crisco Oil Pure Vegetable Oil (J.M. Smucker Company, Orrville, Ohio) or equivalent all soybean oil and a stir bar are placed in a two liter, three necked round bottom flask. The flask is placed on a hot plate and heated to 400° F. (204° C.) while stirring. Air is set to continually bubble through the oil at 1 psi. The oil is heated until viscosity, at ambient temperature (73° F.±3.5° F. and a relative humidity of less than 70%), is between 105,000 and 125,000 cps, as measured by the viscosity method of Section 5 below, and the color is a dark orange-brown. This may take several days at the above stated conditions to achieve. If the amount of oil being heated or size of the flask is varied, the time may also need to be changed as the polymerization depends largely on exposure to air, thus surface area, volume of oil, and air flow are all critical variations. When the polymerized oil has reached its desired viscosity, the hot plate is turned off and the polymerized oil is allowed to reach room temperature before handling.

5. Viscosity Determination Method

The viscosity of the room temperature polymerized oil is measured on a TA Instruments AR-G2 stress-controlled rheometer using Rheology Advantage Instrument software. The rheometer is calibrated as per operating instructions from manufacturer. The rheometer is fitted with a cone and plate geometry using a 40 mm diameter 2° stainless steel cone. The temperature is controlled via a Peltier plate and is set to 25° C. for the duration of the experiment. The point of zero gap for the geometry is determined using the Zero Gap feature in the instrument software. In order to improve resolution at low torque, rotational mapping is performed. During this rotational mapping process, the software rotates the air bearing at a fixed speed, monitoring the torque required to maintain this speed through a full 360° of rotation. The rotational mapping is completed using the precision setting with 3 iterations.

A sample of polymerized oil (approximately 1 gram) is loaded onto the lower Peltier plate. The cone is lowered to the truncation gap (57 microns for the 40 mm 2° cone) and the sample is seen to fill the gap completely. Once the truncation gap is achieved, the sample is trimmed with a flat ended plastic spatula or similar trimming tool to remove any excess sample.

The viscosity determination method is configured in the software with a conditioning step where the temperature is set to 25° C. and the sample is equilibrated for 2 minutes, followed by a stepped flow step in which the shear stress is stepped from 0.1 Pa to 1000 Pa using log spacing and 10 points per decade. A constant time of 30 seconds per point is used, averaging the data over the last 5 seconds at each shear stress point during the stepped flow step of the method.

Under the polymerization conditions described herein, the resulting polymerized oil displayed a zero-rate plateau viscosity followed by shear thinning response when analyzed by this viscosity determination method. This zero-rate viscosity is determined from a fit of the viscosity versus shear rate data using the Cross model. Data below a torque of 3 microN-m is excluded from fitting.

6. 1% Dye in Oil Preparation

A dye is added to the polymerized oil to aid in measuring the oil capture. Weigh into a glass container, 0.5 g±0.05 g Sudan Red G Dye (Sigma Aldrich, St. Louis, Mo.) and 50.0 g±1.0 g polymerized oil (see Method to Polymerize Oil described above). Mix using a spatula to evenly distribute dye into polymerized oil.

7. 2% Dyed Oil in Hexanes Preparation

Weigh into a glass container, 2.0 g±0.025 g 1% dye in oil, prepared above. Volumetrically, add 100 mL of hexanes (Sigma Aldrich, St. Louis, Mo.). Cap the glass container.

Vortex (Vortex Genie 2 or equivalent) vigorously for several minutes to dissolve the polymerized Crisco into hexanes. Glass beads may be added to aid the dissolution process. The solution should be allowed to sit for at least 1 hour on a shaker table set at 200 rpm (VWR mini-shaker 100-1200 rpm or equivalent). If there are non-soluble solids, use a spatula to remove and discard them. Use solution within 24 hours of preparation.

8. Tile Preparation

Obtain glossy white ceramic tiles (3 inch by 6 inch white ceramic tile, US Tile Company manufactured by Roca Tile Company, Miami, Fla. or equivalent). Measure the surface tension of one of the tiles (37±5 dyn/cm nonpolar and 35±5 dyn/cm polar values) as set forth below. If it is not within the range, discard tiles and purchase new tiles.

The surface tension of a tile is measured using a modified ASTM D7490-13 Standard Test Method for Measurement of the Surface Tension of Solid Coatings, Substrates and Pigments using Contact Angle Measurements. The modification to ASTM D7490-13 is that 50.8 dyn/cm (nonpolar) and 0 dyn/cm (polar) are the values used for the diidomethane instead of the values identified in ASTM D7490-13.

Cut a tile from above to 3 inch by 3 inch square. Pipette 1 mL (in two 0.5 mL aliquots, allowing drying between aliquots of no more than 5 minutes) of dyed oil in hexanes solution onto the tile to form the soiled tile for testing. Allow soiled tiles to dry in a hood at room temperature for at least 10 minutes but no more than 30 minutes.

9. Determine Water Loading Level to Add to Test Sample

Weigh substrate to determine how much distilled water to add to the test sample to be tested. Determine the water loading level as follows:

WaterLoadingLevel=Weight_{Substrate}*3.5

10. Swinging Arm Tester Preparation, Sample Loading, and Wiping

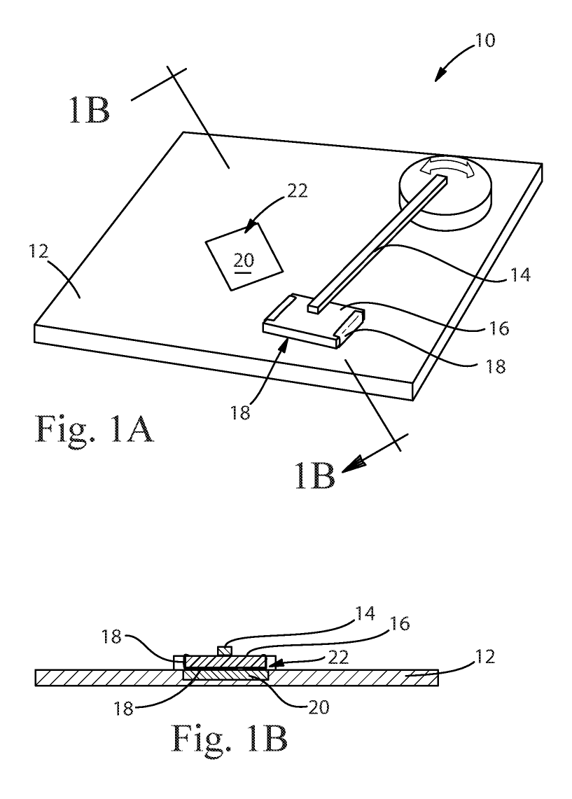

A soiled tile from Step 8 above is then placed on a Swinging Arm Tester 10, as shown in FIGS. 1A and 1B. The Swinging Arm Tester 10 comprises a soiled tile support surface 12 having a recess for completely receiving a soiled tile 20 and a movable arm 14. The movable arm 14 comprises a test sample holder 16 for securely holding without any slack, wrinkles, ripples, or folds the test sample 18 from Step 1 above, such that the test sample 18 covers the entire surface of the test sample holder 16 that moves over the soiled tile 20 during the test. The Swinging Arm Tester 10 needs to be clean and dry before use.

To operate the Swinging Arm Tester 10, place a soiled tile 20 in recess of the soiled tile support surface 12 such that the soiled surface 22 of the soiled tile 20 is exposed to the test sample 18 on the test sample holder 16 during operation of the Swinging Arm Tester 10. The soiled surface 22 needs to be flush with the soiled tile support surface 12.

Then, slowly pipette distilled water (23° C.±2° C.) in the amount of the Water Loading Level determined above in Step 9 onto the test sample 18 and immediately securely attach the moistened test sample 18 to test sample holder 16. Turn the Swinging Arm Tester 10 on such that the swinging arm 14 moves the test sample holder 16 along with the test sample 18 over the soiled surface 22 of the soiled tile 20 (once forward and then immediately once backward to return) at a speed of 20 cycles per minute±10%. Then, remove the soiled tile 20 from the soiled tile support surface 12 and remove the test sample 18 from the test sample holder 16 being careful not to touch the surface of the test sample 18 that contacted the soiled surface 22 of the soiled tile 20.

Allow both the removed soiled tile 20 and the removed test sample 18 to dry completely at 73° F.±3.5° F. and a relative humidity of less than 70%.

11. Measurement

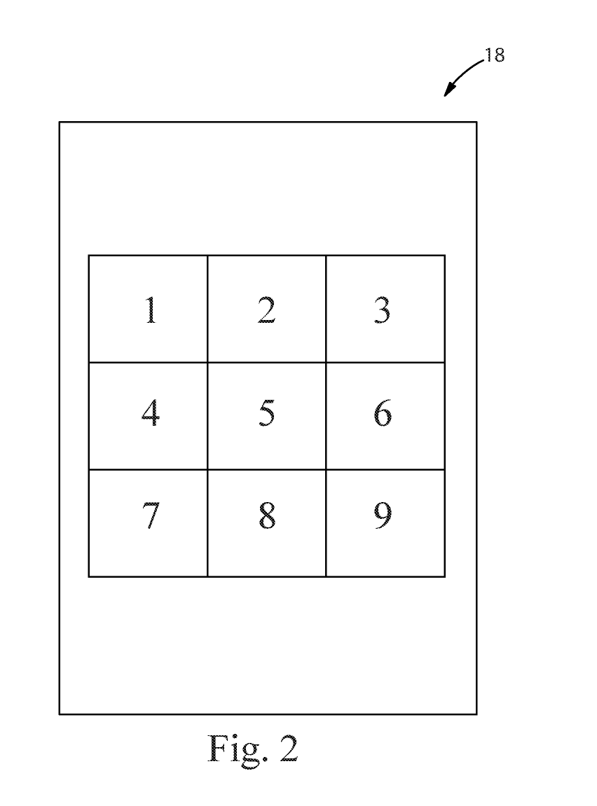

Once the tested test sample 18 from Step 10 has completely dried according to Step 10, the tested test sample 18 is laid out on a flat surface such that no wrinkles, ripples, or folds are present in the areas to be analyzed. Then, a spectrocolorimeter (X-Rite, SpectroEye, Grand Rapids, Mich. or equivalent) is used to take 9 replicate L*a*b readings within equal squares (1-9 shown in FIG. 2) across the tested test sample 18. Each reading should be taken in the approximate center of each of the 9 squares shown in FIG. 2. The “A” portions of the 9 L*a*b readings are summed. N=3 tiles/substrates are run for each polymer sample, for example each oil capture polymer sample, and the average of the N=3 readings is recorded.

The control sample, an “untreated” (containing only 6.7 mL of distilled water, but void an oil capture polymer), dry test sample 18, equivalent to the polymer treated test sample 18 but lacking the polymer treatment, for example oil capture polymer treatment, is measured according to above Steps 9 to 11, as a reference.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A fibrous structure comprising an oil capture polymer such that the fibrous structure exhibits an L*a*b Sum A Value of greater than 85 as measured according to the Oil Capture Test Method.

2. The fibrous structure according to claim 1 wherein the oil capture polymer comprises a monomeric unit derived from a compound selected from the group consisting of: acrylamide, acrylamide derivatives, methacrylamide, methacrylamide derivatives, and mixtures thereof.

3. The fibrous structure according to claim 2 wherein the oil capture polymer comprises greater than 50% by weight of the monomeric unit derived from an acrylamide, acrylamide derivative, methacrylamide, methacrylamide derivative, and mixtures thereof.

4. The fibrous structure according to claim 2 wherein the oil capture polymer comprises greater than 50% by weight of the N,N-dimethylacrylamide and less than 20% by weight of the one or more of the other acrylamide or methacrylamide derivatives.

5. The fibrous structure according to claim 1 wherein the oil capture polymer comprises a homopolymer comprising a monomeric unit selected from the group consisting of: acrylamide, acrylamide derivatives, methacrylamide, and methacrylamide derivatives.

6. The fibrous structure according to claim 5 wherein the oil capture polymer is N, N-dimethylacrylamide.

7. The fibrous structure according to claim 1 wherein the oil capture polymer comprises a cationic monomeric unit.

8. The fibrous structure according to claim 7 wherein the cationic monomeric unit is derived from a cationic monomer selected from the group consisting of: N,N-dimethylaminomethylacrylamide or -methacrylamide, 2-(N,N-dimethylamino)ethylacrylamide or -methacrylamide, 3-(N,N-dimethylamino)propylacrylamide or -methacrylamide, and 4-(N,N-dimethylamino)butylacrylamide or -methacrylamide, 2-(dimethylamino)ethyl acrylate (DMAA), 2-(dimethylamino)ethyl methacrylate (DMAM), 3-(dimethylamino)propyl methacrylate, 2-(tert-butylamino)ethyl methacrylate, 2-(dipentylamino)ethyl methacrylate, and 2(diethylamino)ethyl methacrylate, vinylpyridines, vinylamine, vinylimidazolines, N-vinylformamide, N-vinylacetamide, trimethylammonium propyl methacrylate chloride, trimethylammonium ethylacrylamide chloride, trimethylammonium ethylacrylamide bromide, trimethylammonium methacrylamide chloride, trimethylammonium methacrylamide bromide, trimethylammonium butylacrylamide methyl sulfate, trimethylammonium butylmethacrylamide methyl sulfate, trimethylammonium propylmethacrylamide methyl sulfate, (3-methacrylamidopropyl)trimethylammonium chloride, (3-methacrylamidopropyl)trimethylammonium methyl sulphate, (3-acrylamidopropyl)trimethylammonium chloride, methacryloyloxyethyl-trimethylammonium chloride, methacryloyloxyethyl-trimethylammonium methyl sulfate, acryloyloxyethyltrimethylammonium chloride, 1-ethyl-2-vinylpyridinium bromide, 1-ethyl-2-vinylpyridinium chloride, 1-ethyl-2-vinylpyridinium methyl sulfate, 1-ethyl-4-vinylpyridinium bromide, 1-ethyl-4-vinylpyridinium chloride, 1-ethyl-4-vinylpyridinium methyl sulfate, N,N-dimethyldiallylammonium chloride, dimethylaminopropylmethacrylamide chloride, N-(3-chloro-2-hydroxypropyl)trimethylammonium and 2-hydroxy-N¹-(3-(2((3-methacrylamidopropyl)dimethylammino)-acetamido)propyl)-N¹, N¹, N³, N³, N³-pentamethylpropane-1,3-diaminium chloride, and mixtures thereof.

9. The fibrous structure according to claim 7 wherein the cationic monomeric unit is derived from a cationic monomer selected from the group consisting of: dimethylaminoethyl (meth)acrylate, dimethylaminopropyl (meth)acrylate, di-tert-butylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine and vinyl imidazole, and mixtures thereof.

10. A single-ply or multi-ply sanitary tissue product comprising a fibrous structure according to claim 1.

11. A fibrous structure comprising an oil capture polymer such that the fibrous structure exhibits an L*a*b Sum A Value of greater than 108.0 as measured according to the Oil Capture Test Method.

12. The fibrous structure according to claim 11 wherein the oil capture polymer comprises a monomeric unit derived from a compound selected from the group consisting of: acrylamide, acrylamide derivatives, methacrylamide, methacrylamide derivatives, and mixtures thereof.

13. The fibrous structure according to claim 12 wherein the oil capture polymer comprises greater than 50% by weight of the monomeric unit derived from an acrylamide, acrylamide derivative, methacrylamide, methacrylamide derivative, and mixtures thereof.

14. The fibrous structure according to claim 12 wherein the oil capture polymer comprises greater than 50% by weight of the N,N-dimethylacrylamide and less than 20% by weight of the one or more of the other acrylamide or methacrylamide derivatives.

15. The fibrous structure according to claim 11 wherein the oil capture polymer comprises a homopolymer comprising a monomeric unit selected from the group consisting of: acrylamide, acrylamide derivatives, methacrylamide, and methacrylamide derivatives.

16. The fibrous structure according to claim 15 wherein the oil capture polymer is N, N-dimethylacrylamide.

17. The fibrous structure according to claim 11 wherein the oil capture polymer comprises a cationic monomeric unit.

18. The fibrous structure according to claim 17 wherein the cationic monomeric unit is derived from a cationic monomer selected from the group consisting of: N,N-dimethylaminomethylacrylamide or -methacrylamide, 2-(N,N-dimethylamino)ethylacrylamide or -methacrylamide, 3-(N,N-dimethylamino)propylacrylamide or -methacrylamide, and 4-(N,N-dimethylamino)butylacrylamide or -methacrylamide, 2-(dimethylamino)ethyl acrylate (DMAA), 2-(dimethylamino)ethyl methacrylate (DMAM), 3-(dimethylamino)propyl methacrylate, 2-(tert-butylamino)ethyl methacrylate, 2-(dipentylamino)ethyl methacrylate, and 2(diethylamino)ethyl methacrylate, vinylpyridines, vinylamine, vinylimidazolines, N-vinylformamide, N-vinylacetamide, trimethylammonium propyl methacrylate chloride, trimethylammonium ethylacrylamide chloride, trimethylammonium ethylacrylamide bromide, trimethylammonium methacrylamide chloride, trimethylammonium methacrylamide bromide, trimethylammonium butylacrylamide methyl sulfate, trimethylammonium butylmethacrylamide methyl sulfate, trimethylammonium propylmethacrylamide methyl sulfate, (3-methacrylamidopropyl)trimethylammonium chloride, (3-methacrylamidopropyl)trimethylammonium methyl sulphate, (3-acrylamidopropyl)trimethylammonium chloride, methacryloyloxyethyl-trimethylammonium chloride, methacryloyloxyethyl-trimethylammonium methyl sulfate, acryloyloxyethyltrimethylammonium chloride, 1-ethyl-2-vinylpyridinium bromide, 1-ethyl-2-vinylpyridinium chloride, 1-ethyl-2-vinylpyridinium methyl sulfate, 1-ethyl-4-vinylpyridinium bromide, 1-ethyl-4-vinylpyridinium chloride, 1-ethyl-4-vinylpyridinium methyl sulfate, N,N-dimethyldiallylammonium chloride, dimethylaminopropylmethacrylamide chloride, N-(3-chloro-2-hydroxypropyl)trimethylammonium and 2-hydroxy-N¹-(3-(2((3-methacrylamidopropyl)dimethylammino)-acetamido)propyl)-N¹, N¹, N³, N³, N³-pentamethylpropane-1,3-diaminium chloride, and mixtures thereof.

19. The fibrous structure according to claim 18 wherein the cationic monomeric unit is derived from a cationic monomer selected from the group consisting of: dimethylaminoethyl (meth)acrylate, dimethylaminopropyl (meth)acrylate, di-tert-butylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine and vinyl imidazole, and mixtures thereof.

20. A single-ply or multi-ply sanitary tissue product comprising a fibrous structure according to claim 11.
